(19) United States
(12) Patent Application Publication
(10) Pub. No.: US 2010/0145055 A1
(43) Pub. Date: Jun. 10, 2010

(54) METHOD FOR THE PREPARATION OF SOLIFENACIN

(76) Inventors: Josef Jirman, Praha (CZ); Richard Junek, Praha (CZ)

Correspondence Address: Pearl Cohen Zedek Latzer, LLP, 1500 Broadway, 12th Floor, New York, NY, 10036, US

(21) Appl. No.: 12/520,043

(22) PCT Filed: Dec. 21, 2007
(86) PCT No.: PCT/CZ2007/000119

§ 371 Date: Jan. 17, 2010

(30) Foreign Application Priority Data
Dec. 22, 2006 (CV) PV 2006-828

(51) Int. Cl. C07D 453/00

(52) U.S. Cl. 546/137

(57) ABSTRACT

A method of preparing (1S)-(3R)-1-azabicyclo[2.2.2]oct-3-yl 3,4-dihydro-1-phenyl-2(1H)-isoquinoline carboxylate (solifenacin) or its pharmaceutically acceptable salts with high optic purity, wherein the crude solifenacin base is transformed to the hydrogen tartrate, which is then optionally transformed to another pharmaceutically acceptable salt or the base of solifenacin. A crystalline salt of solifenacin hydrogen tartrate.

### TECHNICAL FIELD

The invention deals with a new method of preparation of (1S)-(3R)-1-azabicyclo[2.2.2]oct-3-yl 3,4-dihydro-1-phenyl-2(1H)-isoquinoline carboxylate of formula III (solifenacin), which is used for symptomatic treatment of urgent incontinence and/or increased frequency of urinating and urgency of urinating in patients with hyperactive urinary bladder.

### BACKGROUND ART

The original patent literature (EP 0 801 067, WO 9620194) describes several methods of preparation of solifenacin.

The first method consists in the reaction of (1S)-ethyl 1-phenyl-1,2,3,4-tetrahydro-2-isoquinoline carboxylate of formula II with 3-(R)-quinuclidol of formula I in a toluene suspension and in the presence of sodium hydride. The reaction mixture is refluxed with simultaneous distillation of the ethanol being formed, which leaves in the form of an azeotropic mixture with toluene.

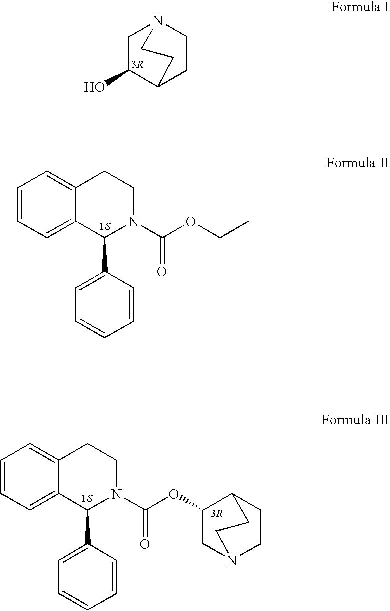

Another method of production described in the patent is the reaction of 1-phenyl-1,2,3,4-tetrahydro-2-isoquinoline carbonyl chloride of formula IV with 3-quinuclidol of formula V in dimethylformamide, producing 1-azabicyclo[2.2.2.]oct-3-yl 3,4-dihydro-1-phenyl-2(1H)-isoquinoline carboxylate of formula VI.

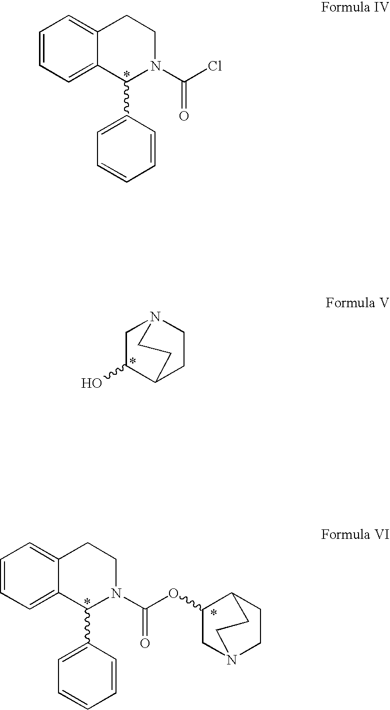

In EP 0 801 067, or WO 9620194 another version is published, where 3-(R)-quinuclidyl alkyl carbonate is reacted with 1-phenyl-1,2,3,4-tetrahydro-2-isoquinoline to produce solifenacin. Crude solifenacin obtained with the use of one of the above mentioned methods of preparation is subsequently isolated as the hydrochloride or oxalate and during their crystallization purification from unreacted starting materials and side products occurs. However, the purifying effect of the transformation to these salts is small. To achieve purity that is suitable for the preparation of the end product, solifenacin succinate, which is used for the preparation of medicaments, the crystallization of HCl or oxalate must be repeated several times, which naturally reduces the yields.

### DISCLOSURE OF INVENTION

The invention provides a new method of preparation of optically pure (1S)-(3R)-1-azabicyclo[2.2.2]oct-3-yl 3,4-dihydro-1-phenyl-2(1H)-isoquinoline carboxylate (solifenacin) or its pharmaceutically acceptable salts, which consists in the preparation of solifenacin hydrogen tartrate in a crystalline form. The invention is based on the exceptionally good purification effect of the transformation of the crude base to the hydrogen tartrate by the action of L-tartaric acid and subsequent isolation of crystalline solifenacin hydrogen tartrate. The method of the invention can be used for purification of solifenacin prepared by any known method of preparation, in particular for solifenacin prepared by reaction of (1S)-alkyl 1-phenyl-1,2,3,4-tetrahydro-2-isoquinoline carboxylate with s 3-(R)-quinuclidol with catalysis of a non-nucleophilic base.

Solifenacin hydrogen tartrate has not been prepared and characterized so far. Crystalline solifenacin hydrogen tartrate is a stable salt that can be transformed to another pharmaceutically acceptable solifenacin salt, e.g. succinate, or used directly for the preparation of a medicament.

This salt has been characterized by the X-ray, DSC, IR and CP-MAS¹³C NMR spectroscopy methods.

The method of the invention can be carried out, e.g., in the following manner:

A reaction mixture with crude solifenacin, prepared by any known method (see above) is cooled after the achievement of the conversion and water is added to the mixture. After separation of the organic phase the aqueous layer is washed with another portion of the organic solvent. The combined organic extracts are washed with water, saline and finally with water again. The organic layer is subsequently evaporated to dryness.

After this purification some impurities still remain in the reaction mixture, such as (1R)-(3R)-1-azabicyclo[2.2.2]oct-3-yl 3,4-dihydro-1-phenyl-2(1H)-isoquino line carboxylate, (1S)-(3S)-1-azabicyclo[2.2.2]oct-3-yl 3,4-dihydro-1-phenyl-2(1H)-isoquinoline carboxylate and 1(S)-phenyl-1,2,3,4-tetrahydroisoquinoline.

As purification of solifenacin by its crystallization in the form of the HCl salt or oxalate has manifested low efficiency, there was an effort to find a more suitable way of purification of crude solifenacin.

What has proved to be more suitable was to convert the solifenacin base to the succinate by the action of succinic acid. However, by far the best cleaning effect was achieved with the conversion of the crude solifenacin base to the hydrogen tartrate crystalline salt by the action of L-tartaric acid.

The crystalline form of the hydrogen tartrate of (1S)-(3R)-1-azabicyclo[2.2.2]oct-3-yl 3,4-dihydro-1-phenyl-2(1H)-isoquinoline carboxylate has been characterized by means of the X-ray diffraction pattern method (signals at 3.9, 11.6, 12.1, 13.9, 17.8, 19.5 and 24.5±0.2 degrees 2θ), IR and CP-MAS¹³C NMR spectroscopies and DSC (1 peak at 200.0° C.) (FIGS. 1 to 4).

The process that has proved to represent the most efficient way of preparation of solifenacin is the process comprising: the preparation of the solifenacin base by reaction of (1S)-alkyl 1-phenyl-1,2,3,4-tetrahydro-2-isoquinoline carboxylate with 3-(R)-quinuclidol with catalysis by a non-nucleophilic base (Scheme 1),

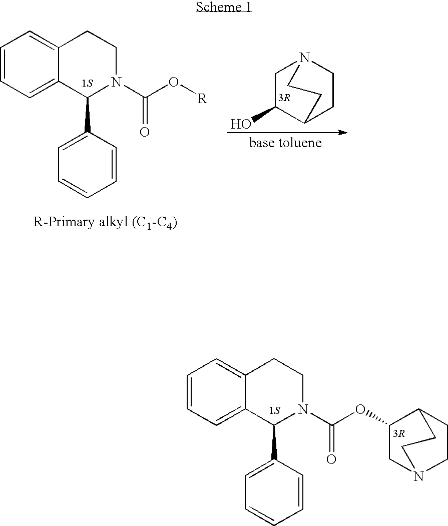

further the isolation of the crude base and subsequent final purification, which consists in transforming the solifenacin base to the hydrogen tartrate by the action of L-tartaric acid in the environment of a polar solvent or mixture of polar solvents, and isolation of crystalline solifenacin hydrogen tartrate.

The solifenacin hydrogen tartrate pure salt prepared this way can be transformed to pharmaceutically commonly used solifenacin succinate, or directly used for the preparation of a medicament.

A non-nucleophilic base is a base having considerably reduced capability of nucleophilic substitution. This is mainly the case of sterically hindered alcoholates or amines, further of lithium compounds or of the group of substances called phosphazenes, in particular e.g. potassium tert-butoxide, sodium tert-butoxide, tert-butyllithium, LDA, K-HMDS, DBU, DBN, 2,6-di-tert-butyl-4-methylpyridine, P1-t-Bu base, BEMP, BTPP and P2-t-Bu base while potassium tert-butoxide and sodium or potassium tert-amylate are especially suitable. From the group of polar solvents for the preparation and isolation of hydrogen tartrate it is most suitable to use C₁-C₄ alcohols, their mixtures and possibly their combinations with water.

Solifenacin tartrate can be alternatively prepared in such a way that L-tartaric acid is added directly to the organic phase with the crude solifenacin base, i.e. without prior isolation of the base, e.g. in such a way that an equivalent of the saturated solution of L-tartaric acid in an alcohol is added to the solution of the crude base in a non-polar solvent. Solifenacin hydrogen tartrate is precipitated, sucked off and subsequently crystallized in a usual way.

### BRIEF DESCRIPTION OF DRAWINGS

FIG. 1 represents an XRPD diffraction pattern of (1S)-(3R)-1-azabicyclo[2.2.2]oct-3-yl 3,4-dihydro-1-phenyl-2(1H)-isoquinoline carboxylate hydrogen tartrate.

FIG. 2 represents an IR spectrum of (1S)-(3R)-1-azabicyclo[2.2.2]oct-3-yl 3,4-dihydro-1-phenyl-2(1H)-isoquinoline carboxylate hydrogen tartrate.

FIG. 3 represents a CP-MAS¹³C NMR spectrum of (1S)-(3R)-1-azabicyclo[2.2.2]oct-3-yl 3,4-dihydro-1-phenyl-2(1H)-isoquinoline carboxylate hydrogen tartrate.

FIG. 4 represents a DSC curve of (1S)-(3R)-1-azabicyclo[2.2.2]oct-3-yl 3,4-dihydro-1-phenyl-2(1H)-isoquinoline carboxylate hydrogen tartrate

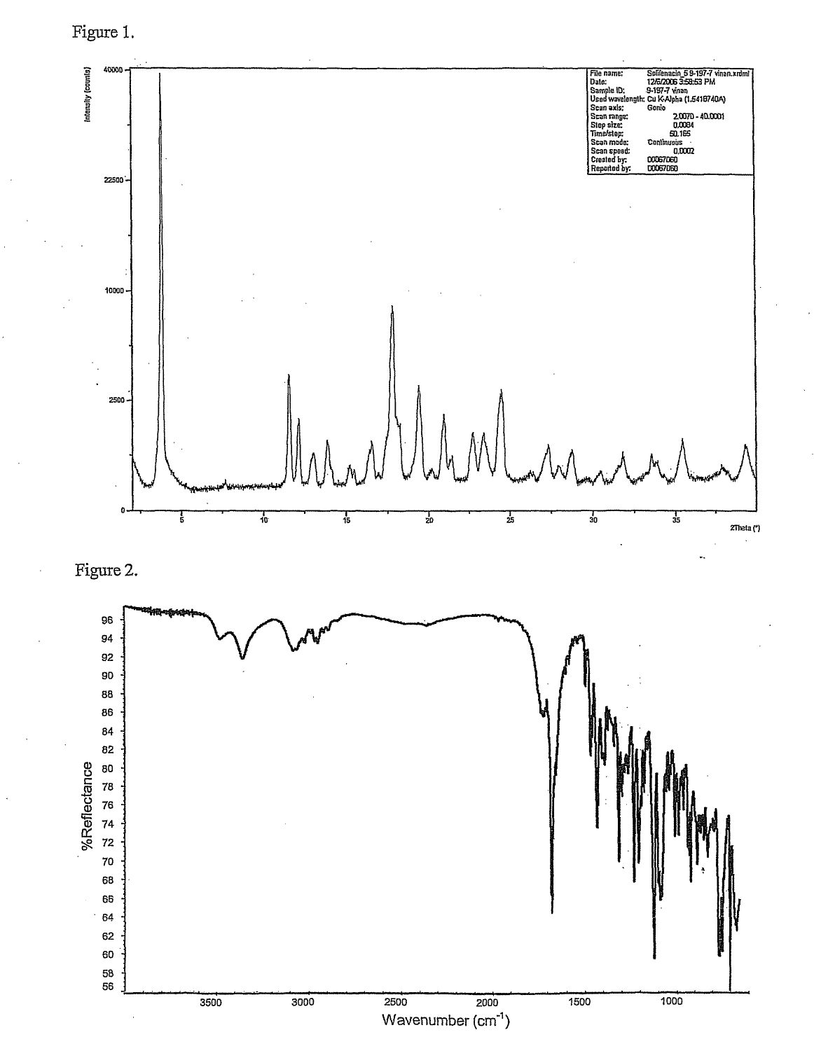

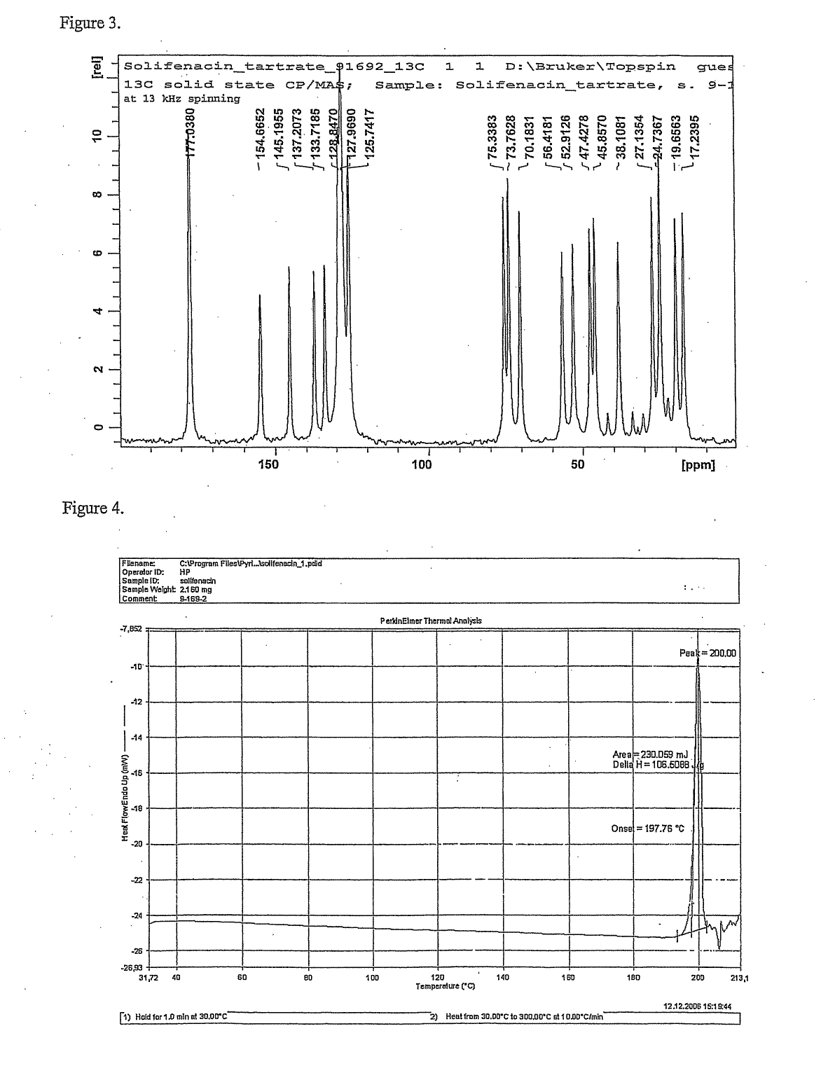

### EXAMPLES

### General Procedure of Examples 1-6

3(R)-Quinuclidol (0.1-0.15 mmol) is added to a solution of 1(S)-ethyl 1-phenyl-1,2,3,4-tetrahydro-2-isoquinoline carboxylate (0.1 mmol) in toluene (25 ml). After heating to 90° C. and complete dissolution of both starting substances potassium tert-butanolate (0-0.05 mmol) is added to the mixture during continuous stirring and the resulting fine suspension is heated up to boil. The azeotropic mixture toluene—alcohol is gradually removed from the mixture by distillation. The reaction is completed after the achievement of the corresponding conversion.

The course of the reaction (substance conversion) was monitored with gas chromatography.

The optical purity of the product was determined with capillary electrophoresis.

The results of analyses of the products prepared in accordance with examples 1-6 are summarized in the table.

| | Charge (%) | | 3 hours (%) | | | 4 hours (%) | | | 5 hours (%) | | | 6 hours (%) | | | 10 hours (%) | | |
|---|---|---|---|---|---|---|---|---|---|---|---|---|---|---|---|---|---|
| Batch | Chin | tBOK | conv. | R,R | S,S | conv. | R,R | S,S | conv. | R,R | S,S | conv. | R,R | S,S | conv. | R,R | S,S |
| 9-142 | 120 | 20 | 96.11 | 1.77 | 1.97 | 96.45 | 2.07 | 2.27 | 97.07 | 2.27 | 2.27 | — | — | — | — | — | — |
| 9-143 | 110 | 10 | 51.29 | 0.21 | 0.50 | 78.20 | 1.32 | 0.78 | 87.45 | 1.82 | 0.95 | 95.86 | 5.12 | 1.42 | 96.07 | 5.13 | 1.51 |
| 9-144 | 110 | 20 | 96.80 | 5.12 | 2.33 | — | — | — | — | — | — | — | — | — | — | — | — |
| 9-145 | 130 | 20 | 87.52 | 2.15 | 0.78 | — | — | — | — | — | — | — | — | — | — | — | — |
| 9-146 | 120 | 30 | 96.01 | 4.56 | 1.38 | — | — | — | — | — | — | — | — | — | — | — | — |
| 9-148 | 130 | 10 | 34.53 | 0.00 | 0.00 | — | — | — | 64.93 | 0.40 | 0.48 | 76.45 | 0.58 | 0.75 | — | — | — |

### Example 7

4 g of the solifenacin base prepared in accordance with procedure 6, which contained 4.5% of (1R)-(3R)-1-azabicyclo[2.2.2.]oct-3-yl 3,4-dihydro-1-phenyl-2(1H)-isoquinoline carboxylate, 1.4% of (1S)-(3S)-1-azabicyclo[2.2.2]oct-3-yl 3,4-dihydro-1-phenyl-2(1H)-isoquinoline carboxylate and 4.0% of 1(S)-phenyl-1,2,3,4-tetrahydroisoquinoline were dissolved in 30 ml of methylethylketone. An equivalent of a 3M solution of hydrogen chloride in methanol was added to the solution and the mixture was left to freely crystallize at the room temperature. After sucking off and washing, 42% of solifenacin hydrochloride was obtained, which contained 1.7% of (1R)-(3R)-1-azabicyclo[2.2.2]oct-3-yl 3,4-dihydro-1-phenyl-2(1H)-isoquinoline carboxylate hydrochloride, 0.6% of (1S)-(3S)-1-azabicyclo[2.2.2]oct-3-yl 3,4-dihydro-1-phenyl-2(1H)-isoquinoline carboxylate hydrochloride and 1.3% of 1(S)-phenyl-1,2,3,4-tetrahydroisoquinoline hydrochloride. In crystallizing the product from methylethylketone 58% of solifenacin hydrochloride was obtained, which contained 0.9% of (1R)-(3R)-1-azabicyclo[2.2.2]oct-3-yl 3,4-dihydro-1-phenyl-2(1H)-isoquinoline carboxylate hydrochloride, 0.4% of (1S)-(3S)-1-azabicyclo[2.2.2]oct-3-yl 3,4-dihydro-1-phenyl-2(1H)-isoquinoline carboxylate hydrochloride and 0.7% of 1(S)-phenyl-1,2,3,4-tetrahydroisoquinoline hydrochloride. After the second crystallization from methylethylketone 63% of solifenacin hydrochloride was obtained, which contained 0.4% of (1R)-(3R)-1-azabicyclo[2.2.2]oct-3-yl 3,4-dihydro-1-phenyl-2(1H)-isoquinoline carboxylate hydrochloride, 0.18% of (1S)-(3S)-1-azabicyclo[2.2.2.]oct-3-yl 3,4-dihydro-1-phenyl-2(1H)-isoquinoline carboxylate hydrochloride and 0.3% of 1(S)-phenyl-1,2,3,4-tetrahydroisoquinoline hydrochloride.

### Example 8

3(R)-Quinuclidol (44.3 mmol) was added to a solution of 1(S)-ethyl 1-phenyl-1,2,3,4-tetrahydro-2-isoquinoline carboxylate (36.7 mmol) in 250 ml of toluene. After heating to 90° C. and complete dissolution of both starting substances potassium tert-amylate (7.4 mmol) was added to the mixture during continuous stirring and the resulting fine suspension was heated up to boil. The azeotropic mixture toluene—alcohol was gradually removed from the mixture by distillation. The reaction was finished after 3 hours of boiling, when the conversion in accordance with GC achieved 96%. Water (100 ml) was added to the mixture and the mixture was stirred at the laboratory temperature for 30 minutes. After the separation of the toluene layer in a separator the aqueous layer was washed with toluene (2×100 ml). The combined organic extracts were then further extracted with 150 ml of diluted hydrochloric acid (6.1 mmol) and the toluene layer was then washed with water (2×50 ml). Then, the aqueous extracts were alcalinized with potassium carbonate (10.9 mmol) and extracted with toluene (3×100 ml). The organic extracts were washed with 100 ml of water, 50 ml of saline, 50 ml of water and evaporated in a rotational vacuum evaporator 12.7 g of the solifenacin base was obtained that contained the following impurities (CE analysis).

3.2% of (1R)-(3R)-1-azabicyclo[2.2.2]oct-3-yl 3,4-dihydro-1-phenyl-2(1H)-iso quinoline carboxylate, 1.1% of (1S)-(3S)-1-azabicyclo[2.2.2]oct-3-yl 3,4-dihydro-1-phenyl-2(1H)-isoquinoline carboxylate and 6.0% of 1(S)-phenyl-1,2,3,4-tetrahydroisoquinoline.

### Example 9

One half of the solifenacin base prepared in accordance with procedure 8 was dissolved in 50 ml of a methanol:2-propanol (1:1) mixture, an equivalent of L-tartaric acid in a methanol:2-propanol (1:1) mixture was added and the mixture was left to freely crystallize at the room temperature. After sucking off and washing, 79% of solifenacin hydrogen tartrate was obtained, which contained 1.1% of (1R)-(3R)-1-azabicyclo[2.2.2.]oct-3-yl 3,4-dihydro-1-fenyl-2(1H)-isoquinoline carboxylate hydrogen tartrate, 0.6% of (1S)-(3S)-1-azabicyclo[2.2.2]oct-3-yl 3,4-dihydro-1-phenyl-2(1H)-isoquinoline carboxylate hydrogen tartrate and no 1(S)-phenyl-1,2,3,4-tetrahydroisoquinoline. In crystallizing the product from the methanol:2-propanol (3:4) mixture 94% of solifenacin hydrogen tartrate was obtained, which contained 0.6% of (1R)-(3R)-1-azabicyclo[2.2.2.]oct-3-yl 3,4-dihydro-1-fenyl-2(1H)-isoquinoline carboxylate hydrogen tartrate and 0.4% of (1S)-(3S)-1-azabicyclo[2.2.2]oct-3-yl 3,4-dihydro-1-phenyl-2(1H)-isoquinoline carboxylate hydrogen tartrate. After the second crystallization from ethanol 89% of solifenacin hydrogen tartrate was obtained, which did not contain any detectable impurities. The product was characterized by means of the following analytical methods: X-ray, IR and CP-MAS¹³C NMR spectroscopies and DSC.

### Example 10

The other half of the solifenacin base prepared in accordance with procedure 8 was dissolved in 15 ml of 2-propanol, an equivalent of succinic acid in 55 ml of 2-propanol was added and the mixture was left to freely crystallize at the room temperature. After sucking off and washing, 66% of solifenacin succinate was obtained, which contained 1.0% of (1R)-(3R)-1-azabicyclo[2.2.2.]oct-3-yl 3,4-dihydro-1-phenyl-2(1H)-isoquinoline carboxylate succinate, 0.7% of (1S)-(3S)-1-azabicyclo[2.2.2.]oct-3-yl 3,4-dihydro-1-phenyl-2(1H)-isoquinoline carboxylate succinate and 0.3% of 1(S)-phenyl-1,2,3,4-tetrahydroisoquinoline. In crystallizing the product from 2-propanol 86% of solifenacin succinate was obtained, which contained 0.41% of (1R)-(3R)-1-azabicyclo[2.2.2]oct-3-yl 3,4-dihydro-1-phenyl-2(1H)-isoquinoline carboxylate succinate, 0.35% of (1S)-(3S)-1-azabicyclo[2.2.2.]oct-3-yl 3,4-dihydro-1-phenyl-2(1H)-isoquinoline carboxylate succinate and 0.1% of 1(S)-phenyl-1,2,3,4-tetrahydroisoquinoline. After the second crystallization from 2-propanol 89% of solifenacin succinate was obtained, which contained 0.17% (1R)-(3R)-1-azabicyclo[2.2.2]oct-3-yl 3,4-dihydro-1-phenyl-2(1H)-isoquinoline carboxylate succinate, 0.33% of (1S)-(3S)-1-azabicyclo[2.2.2]oct-3-yl 3,4-dihydro-1-phenyl-2(1H)-isoquinoline carboxylate succinate and 0.07% of 1(S)-phenyl-1,2,3,4-tetrahydroisoquinoline.

1. A method for the preparation of (1S)-(3R)-1-azabicyclo[2.2.2]oct-3-yl 3,4-dihydro-1-phenyl-2(1H)-isoquinoline carboxylate (solifenacin) or its pharmaceutically acceptable salts with high optic purity, wherein the crude solifenacin base is converted to the hydrogen tartrate, which is then converted to another pharmaceutically acceptable salt or the base of solifenacin.

2. The method in accordance with claim 1, wherein the solifenacin base is converted to the hydrogen tartrate by the action of 1-tartaric acid and crystalline solifenacin hydrogen tartrate is subsequently isolated.

3. The method in accordance with claim 2, wherein the solifenacin hydrogen tartrate is further re-crystallized from a polar solvent or a mixture of polar solvents, selected from the group of C₁-C₄ alcohols, or their combination with water.

4. The method in accordance with claim 2, wherein the crude solifenacin base is prepared by reaction of (1S)-alkyl 1-phenyl-1,2,3,4-tetrahydro-2-isoquinoline carboxylate, where alkyl means a primary C₁-C₄ alkyl, with 3-(R)-quinuclidol in the environment of a non-nucleophilic base.

5. The method in accordance with claim 4, wherein a non-nucleophilic base from the group of sterically hindered alcoholates or amines, or lithium compounds, or phosphazenes, is used.

6. The method in accordance with claim 5, wherein potassium, sodium or lithium tert-butanolate is used.

7. The method in accordance with claim 5, wherein potassium or sodium tert-amylate is used.

8. A crystalline salt of solifenacin hydrogen tartrate.

9. The crystalline salt in accordance with claim 8, characterized by the x-ray spectrum record with typical signals at: 3.9, 11.6, 12.1, 13.9, 17.8, 19.5 and 24.5±0.2 degrees 2θ.

10. The crystalline salt in accordance with claim 8, characterized by the dsc record with 1 endothelin with the peak at 200.0° C. at the heating speed of 10° c/min.

11. The crystalline salt in accordance with claim 8, characterized by the signals at 11.2 19.6 24.7 27.1 38.1 45.8 47.4 52.9 56.4 70.1 73.7 75.3 125.7 127.9 128.8 133.7 137.2 145.2 154.6 177.0 ppm in the CP/13C MAS NMR spectrum.
